(19) United States
(12) Patent Application Publication
(10) Pub. No.: US 2002/0123789 A1
(43) Pub. Date: Sep. 5, 2002

(54) Stent cover

(76) Inventors: Ralph T. Francis, New Brighton, MN (US); Qing Hong Zhao, Andover, MN (US); B. Nicholas Oray, Woodbury, MN (US); Anne E. Metzger, Alexandria, VA (US)

Correspondence Address: Philip M. Goldman, FREDRIKSON & BYRON, P.A., 1100 International Centre, 900 Second Avenue South, Minneapolis, MN, 55402-3397, US

(21) Appl. No.: 09/872,482

(22) Filed: May 31, 2001

Related U.S. Application Data
(63) Continuation of application No. PCT/US98/25674, filed on Dec. 4, 1998.

(51) Int. Cl. A61F 2/06

(52) U.S. Cl. 623/1.13; 623/1.41; 623/916

(57) ABSTRACT

A stent cover useful as a barrier between an expandable stent and the vascular surface. The cover provides an optimal combination of such properties as thickness, physical characteristics and biocompatability. The cover can be formed to and positioned upon the size and shape of the unexpanded stent, and to then be expanded in situ upon expansion of the stent itself. The cover is prepared from natural tissues such as umbilical arteries, bovine pericardium, and porcine peritoneum.

### CROSS-REFERENCE TO RELATED APPLICATIONS

The present application is a continuation of and claims priority to International Application No. PCT/US98/25674 (published as International Publication No. WO 00/33768), filed Dec. 4, 1998 and designating the United States, the entire disclosure of which is incorporated herein by reference.

### TECHNICAL FIELD

The present invention relates to stents and to methods for reducing the occurrence of restenosis, and particularly restenosis due to intimal hyperplasia, in the course of the use of such stents. In a related aspect, the invention relates to coatings and covers for use on stents. In a further related aspect, the invention relates to methods of preparing and methods of using coated or covered stents.

### BACKGROUND OF THE INVENTION

A number of improvements and advances have been made in recent years in the technique of percutaneous transluminal coronary angioplasty (“PTCA”). In turn, intravascular stent implantation has arisen to address the need to treat abrupt vessel closure and prevent restenosis after angioplasty. Coronary stents represent the first major breakthrough in interventional cardiology since the introduction of balloon angioplasty in the 1970s. Before the use of stents, 30% to 40% of patients who had a coronary interventional procedure were likely to experience restenosis. Restenosis rates have dropped from 10% to 20% of all stented procedures in recent clinical studies, prompting rapid adoption of coronary stents among the interventional cardiology community.

The cardiovascular stent market reached an approximate level $2 billion in annual sales by the year 1998, with further growth expected. See L. Haimovitch, Cardiovascular Device Update 4(11):1-5, November 1998. The companies having stents in development or already marketed include Johnson & Johnson Interventional Systems/Cordis, Medtronic, Pfizer/Schneider and Arterial Vascular Engineering, along with AngioDynamics, Biotronik, Boston Scientific Corporation, C. R. Bard, Cook, Inc., Devon Medical, Global Therapeutics, Guidant Corporation, and Progressive Angioplasty Systems.

Restenosis, however, continues to pose a problem for cardiologists. Restenosis is typically viewed as involving two components—recoil (collapse of the artery after it's been opened) and the proliferative response (development of scar tissue). While stents prevent recoil, they are not presently able to inhibit scar formation. In fact, through unavoidable injury to the vessel surface, stents may actually increase the formation of scar tissue. A number of approaches have been suggested for tackling the problem of restenosis, including the use of coronary radiation to inhibit proliferation.

See, for instance, Donald F. Phillips, JAMA, Medical News & Perspectives—May 1, 1996, “New Ideas on Pathology of Restenosis”, which describes the manner in which the long-term benefits of PTCA, as a means of treating coronary artery disease, remain tempered by restenosis, which develops in 30% to 50% of patients within 6 months after the procedure is done. The prevailing view is that the angioplasty procedure damages the coronary wall, leading to platelet activation, which causes vascular smooth muscle cells to proliferate and migrate to the damaged area of the vessel where they accumulate. This creates an extracellular matrix that forms an intimal lesion, resulting in the loss of lumen cross-sectional area, thus impeding blood flow. The Phillips article goes on to suggest that the elimination of restenosis may require a combined approach, including a mechanical device to resist geometric remodeling and a pharmacologic agent to inhibit cellular proliferation.

Along these lines, a variety of approaches have been described for preventing cell ingrowth and intimal hyperplasia, including modifications to the stent design itself, as well as the use of coatings and coverings. In particular, a variety of approaches and materials have been described for use in covering stents. Fouere (WO 9816172), for instance, discloses a balloon-expandable stent which can also be bent in the center to fit straight or curved vessels. The stent includes a laser-machined thin metal tube with radially-expandable mesh at ends and central zigzag links, with a biocompatible covering. The covering is described as being a flexible synthetic material such a polyurethane, silicone rubber or polyester, in order to prevent endothelium with penetration through the mesh and into the tube.

Chu et al. (International Patent Application No. WO 9800090, assigned to Baxter) describe a tubular stented graft, alternately used in radially compact and radially expanded configurations having first and second diameters respectively. The stent graft includes a continuous, tubular polytetrafluoroethylene covering formed on a stent comprising lateral openings in the stent when the stent is at the radially expanded diameter. The polytetrafluoroethylene covering includes a tubular outer layer formed of expanded, sintered polytetrafluoroethylene. The outer layer is about the outer surface of the stent so that the stent is captured between the outer layer and a tubular base graft, formed of expanded sintered polytetrafluoroethylene.

Solovay, in U.S. Pat. No. 5,769,884 (assigned to Cordis), describes a controlled porosity endovascular implant for damaged or stenosed blood vessel. The implant includes a stent having an expandable frame structure and a stent covering with two regions having different porosities.

Buirge et al. (U.S. Pat. No. 5,693,085, assigned to Scimed Life Systems) describes a vascular prosthesis that includes a stent with a collagen sleeve, with the collagen in biased stretch orientation to the stent. The patent includes a combination of a vascular prosthesis comprising an expandable support framework stent, and a covering sleeve and/or liner of a collagen material.

Finally, House et al. (U.S. Pat. No. 5,620,763, assigned to Gore & Assoc.) describes a thin-walled, porous, seamless plastic tube for use as an intraluminal vascular graft or as covering for intraluminal stent. The tube may have a wall thickness ranging from less than 0.1 mm to less than 0.06 mm, preferably about 0.2 mm. Also claimed is a tube formed by two layers of 0.05 mm thick membrane with a 0.013 thick non-porous layer of fluorinated ethylene propylene therebetween. The PTFE layers are oriented with their fibrils disposed perpendicularly, and the tube formed as hereinbefore described.

The use of stent covers is generally limited by a variety of considerations, including the need to find a cover material that is sufficiently biocompatible. Such materials should also be sufficiently thin, so as not to add undue size to the already-limited stent, yet strong enough for its intended purpose. The ability to achieve a thin material, however, is often tempered by the accompanying lack of strength or integrity.

A limited number of biological materials have also been used as well, or proposed for use as stent covers. See, for instance, H. B. Lin et al. Proceedings of the 24^{th} Annual Meeting of the Society for Biomaterials, Apr. 22-26, 1998, and Vanderwalde et al., Stent-Graft (SG) with Bovine Pericardium (BP): Acute Results in Porcine Coronary Arteries, TCT-14 (abstract) American J. Cardiol., pp 5S-6S, October, 1998. The Vanderwalde et al. work is said to employ a “thin BP” obtained by “special processing”. The abstract itself provides little detail, and speaks only of a process that includes obtaining pericardium and suturing it into a tube that is then positioned onto a stent, and attached to the stent using sutures. There is no mention of the tissue being treated in any manner, or of the formed tube being closely adapted to the constricted size of the stent. The abstract implies that the tube is simply formed with a diameter sufficient to accommodate the expanded diameter of the stent, and is then retained in place on the constricted stent by the sutures described. Nor is there any indication of the actual thickness of the “thin” material, or the manner in which the “special processing” may have been used to achieve such a material. Assuming it can be determined that such processing involved peeling the pericardium, in the hydrated state, a comparative example herein shows that such a method provides an inferior combination of absolute thickness, uniformity of thickness, strength, and consistency of results for use in commercial applications.

What is clearly needed, therefore, are materials and methods for reducing or eliminating intimal hyperplasia that provide an optimal combination of such properties as efficacy, adaptability to various stent designs, availability and cost. With regard to the use of biological tissues for such purposes, what is particularly needed are materials that provide these and other features, together with an improved combination of absolute thickness, as well as consistency and uniformity in thickness.

### SUMMARY OF THE INVENTION

The present invention provides a stent cover adapted to act as a barrier between an expandable vascular stent and the vascular surface. The stent cover is adapted to be placed over a stent (e.g., an expandable metallic stent) that is deployed percutaneously within a mammalian vein or artery. The stent cover functions to increase the patency rate of the vein or artery after stent placement, and to decrease the incidence and/or extent of intimal hyperplasia, as compared to an identical stent lacking such a cover.

In another aspect, the present invention provides a stent cover comprising a natural tissue dimensioned and adapted to be placed over an expandable stent and to be expanded in situ, upon expansion of the stent, in a manner that permits the cover to maintain its integrity and decrease the incidence and/or extent of intimal hyperplasia, as compared to a stent lacking the cover. The stent cover also provides an improved combination of absolute thickness, uniformity of thickness and physical characteristics (e.g., strength) as compared to a pericardium cover prepared by other routes.

The stent cover can be formed (e.g., crimped) to assume substantially the shape of the constricted stent, and thereafter expanded, upon expansion of the stent, to assume the shape and diameter of the expanded stent as well. Preferably, the expansion is largely achieved by physical means, as opposed to elasticity of the material itself, e.g., by crimping the cover onto the constricted stent and permitting it become uncrimped as the stent is expanded. Optionally, however, the material can contribute an element of elasticity as well, particularly in its expanded form, for instance, to accommodate slight variations in size, and to form a more intimate fit.

In a preferred embodiment, the natural tissue is a strong tissue of mammalian origin and is either itself sufficiently thin and biocompatible for its intended purpose, or can be rendered so in the manner described herein. The word “thin”, and inflections thereof, as used in describing a stent cover of this invention, refers to a tissue that imparts negligible additional size to the stent upon insertion, or when expanded, such that the stent can be delivered in a normal fashion and with undue trauma or damage to the vessel. Many, if not all, stent covers described previously, tend to be unduly thick, or lack uniformity, when used in this respect.

By comparison, Applicants have discovered that natural tissues can be obtained and/or prepared to be sufficiently thin. In a preferred embodiment, for instance, Applicants have discovered that even bovine pericardium can be prepared in a particularly thin and uniform fashion, e.g., by a method that involves at least one, and preferably two or three, “dry peeling” steps as described herein (as opposed to a single peeling in the wet or hydrated state). A preferred method of the present invention is a multi-step method that includes, first, carefully “dry peeling” the vacuum dried tissue, e.g., to a thickness of about 100 microns or more (and preferably between about 150 microns to 250 microns thickness), followed by a second step in which the tissue is rehydrated and then air dried to provide a compact tissue, having a final thickness of about one-third the thickness of the dry-peeled tissue, that is, between about 50 microns and about 100 microns in thickness. In other words, air drying the tissue serves to compact it to on the order of about one-fourth to one-half, and preferably about one-third of its thickness. This can be compared with a thickness of about 200 microns to about 250 microns, which is typically the smallest thickness that can be achieved by a single peeling of the tissue in its hydrated state. Moreover, peeling in the dry state permits the manufacturer to make multiple peels of the tissue, as compared to the single peel typically permitted using hydrated tissue.

In a further preferred embodiment Applicants include yet another step in the processing of pericardium. This step includes treating the tissue with a disinfecting agent, e.g., sodium hydroxide, in order to further lessen the already minimal possibility of bovine spongiform encephalitis (BSE) infection. Such treatment is not only effective as an treatment effective to reduce/eliminate BSE infectivity when used in this manner, but moreover, that a tissue thus treated provides improved or comparable properties as compared to untreated tissues.

Particularly preferred tissues are selected from the group consisting of mammalian arteries (e.g., human umbilical arteries), and fibro-serous and serous membranes, including fibro-serous membranes such as pericardium (e.g., bovine pericardium) and serous membranes such as peritoneum (e.g., porcine peritoneum). Such tissues are preferably obtained, prepared, and/or treated in a manner that renders the tissues biocompatible, e.g., substantially nonantigenic. By the term “substantially nonantigenic” it is meant that the tissue does not elicit an antigenic or other physiological response on the part of the host, to an extent that would render the cover unsuitable for its intended use. A stent cover of this invention can either be permanent (that is, present for so long as the stent itself remains in place), or temporary (e.g., removable or biodegradable over a period of weeks, months or years). Optionally, in turn, such tissues can also be decellularized and/or cross-linked.

A cover of the present invention can be provided in any suitable form, e.g., as flat or textured sheets, strips or in tubular form. Preferred covers are either naturally occurring or prepared (e.g., sutured and/or sealed) to be tubular in shape. Particularly preferred covers are provided in a seamless tubular configuration, e.g., using mammalian vessels such as human umbilical arteries or veins.

A stent cover of this invention is also sufficiently expandible to permit it to be placed upon a stent prior to implantation, and once implanted, to expand with the stent in order to provide a sufficient barrier to intimal hyperplasia. Expandability can be achieved in any suitable fashion, e.g., by the use of a tissue having sufficient elasticity to permit it to be stretched and placed over the unexpanded stent, and to then maintain its integrity by itself expanding upon expansion of the stent itself. In another, and preferred embodiment, the tissue can be positioned upon the stent by crimping, or otherwise conforming it to the outer dimensions of the unexpanded stent. Once positioned within the vein or artery, the combination of hydration and/or stent expansion permit the cover to become uncrimped, and return to substantially its original dimensions. Such a cover is therefore provided with dimensions sufficient to permit it to stay in position upon an expanded stent of predetermined configuration and dimensions.

In a further preferred embodiment, a stent cover of this invention is thin, e.g., sufficiently thin to permit it to be temporarily and reversibly crimped or folded onto a stent, and to then return to substantially its original dimensions upon expansion of the stent in situ. For instance, a particularly preferred cover of the present invention is provided in the form of a cylinder of tissue constructed from thin bovine pericardium which is crimped into an expandable stent cover, and there crosslinked. Crosslinking at that point provides a number of benefits, including biocompatability and permanence in situ, as well as facilitating the ability of the cover to retain its crimped configuration. In other words, in such an embodiment, the overlapping or crimped portions of tissue are themselves crosslinked to other portions of tissue. This crosslinking is of sufficient strength to improve the stability of the crimps during storage and insertion, while also permitting the crimps to become unfolded in situ upon expansion of the stent. Applicants have developed a method for providing bovine pericardium that is particularly thin, as compared to the original tissue, yet surprisingly maintains most or all of its other desired properties, such as strength, integrity, and biocompatability.

In another aspect, the invention provides a combination comprising a stent covered with a natural, optionally treated, tissue of the type described herein. In yet another aspect, the invention provides a method of fabricating a stent cover, and a cover prepared by such a method, as well as a kit comprising a stent and a cover, as described herein, adapted to be positioned on the stent, as well as a covered stent prepared using such a kit. In a further aspect, the invention provides a method of using a stent having a cover as described herein, as well as an artery having an expanded covered stent positioned therein.

### DETAILED DESCRIPTION

Tissues suitable for use as a stent cover of the present invention provide an optimal combination of such properties as availability, biocompatability, strength, the ability to be easily fabricated and used.

Tissues can be obtained from any suitable source, e.g., from mammalian tissues such as arteries as well as serous and fibro-serous membranes. In a particularly preferred embodiment, the tissue source is selected from the group consisting of bovine pericardium, human umbilical tissue (e.g., artery) and porcine peritoneum. Tissues are preferably fixed, e.g., by crosslinking, in order to improve their biocompatability. Suitable crosslinking agents include, for instance, aldehydes such as glutaraldehyde, epoxides, isocyanates, carbodiimides, isothiocyanates, glycidalethers, and acyl azides. Tissues can be fixed at any suitable point, e.g., prior to or after being cleaned, formed, or positioned upon a stent or mandrel. In a preferred embodiment, for instance using pericardium, the tissue is crosslinked after it has been positioned and crimped onto the stent itself, or a suitable mandrel (e.g., one dimensioned to permit the cover to be removed and placed onto a stent of choice).

In one particularly preferred embodiment, the cover is formed from bovine pericardiun, in a method as described herein, to provide the tissue with an optimal combination of biocompatability, thickness, and other physical and physiological properties.

Tissues useful as stent covers of this invention provide an optimal combination of chemical, physical and physiological (e.g., immunological) properties for use as stent covers. In a preferred sense, the tissues provide an optimal combination of such properties as suture retention, shrink temperature, circumferential tensile strength, and tensile strength, as each are determined and described herein. For instance, with regard to suture retention, particularly preferred tissues provide between about 10 g to about 200 g, and more preferably between about 30 g and about 150 g, suture retention. With regard to shrink temperature, preferred tissues provide shrink temperatures between about 70 C. and 90 C., and preferably between about 80 and about 90 C. With regard to circumferential tensile strength, preferred tissues provide between about 0.2 N/mm to about 0.5 N/mm, and more preferably between about 0.3 N/mm and about 0.4 (N/mm). Finally, preferred tissues provide tensile strengths of between about 5 MPa and about 15 MPa, and more preferably between about 7 MPa and about 12 MPa.

Stent covers of the present invention can be fabricated in any suitable shape or configuration, and in any suitable dimensions for their intended use. For instance, the tissue can be provided and packaged in flat (e.g., sheet or tape-like) or tubular form, with either or both major surfaces thereof being optionally textured or modified (e.g., by the covalent attachment, entrapment, and/or adsorption of biologically active factors, lubricious agents, antimicrobial agents, and the like).

See, for instance, M. Valente, et al., “Detoxified Glutaraldehyde Cross-linked Pericardium: Tissue Preservation and Mineralization Mitigation in a Subcutaneous Rat Model”, (J. Heart Valve Dis. 1998 May;7(3):283-91), and C. Stacchino et al., “Detoxification Process for Glutaraldehyde-treated Bovine Pericardium: Biological, Chemical and Mechanical Characterization”, J Heart Valve Dis 1998 Mar;7(2):190-4, the disclosures of each of which are incorporated herein by reference. These articles describe the manner in which glutaraldehyde promotes calcification by the action of toxic aldehyde group residuals from cross-linking. The authors have found that post-fixation treatment with homocysteic acid (HA), besides bonding aldehyde groups and neutralizing toxicity, can enhance biocompatibility due to the strongly electronegative sulfonic group. Moreover, the tissue can be provided with markings or other suitable means to indicate its preferred orientation or direction.

The present stent covers can also be provided to have any desired dimensions, in both their constricted (e.g., crimped) and expanded (e.g., unconstricted) form. Suitable covers, for instance, can be provided having an overall length of between about 5 mm and about 50 mm, preferably between about 10 mm and about 30 mm, and a maximum (e.g., swelled or unconstricted) diameter range between about 1 mm and about 10 mm, preferably between about 2 mm and about 5 mm. Such covers are also able to be constricted (e.g., by crimping or shrinking) to a diameter of between about ½ mm and about 5 mm, and preferably of between about 1 mm and about 3 mm. Particularly preferred are thin covers, e.g., those having an average maximum wall thickness of between about 20 microns and about 80 microns, and preferably between about 40 and about 60 microns

When used in tubular form, for instance, the stent cover can be either seamless or seamed, and is typically adapted to be positioned over a stent of a particular size or size range. The stent cover can be positioned upon the stent in any suitable manner as well, e.g., it can be stretched so as to allow the cover to be expanded, placed over the stent, and allowed to return toward its original dimensions. Tissues can be formed into tubes, for instance, by sealing a flat tissue in a cylindrical form, e.g., by the use of sutures, or in a sutureless fashion as by the use of an adhesive.

Alternatively, or in addition, the stent cover can be crimped (and optionally secured or dried) onto the stent, to be uncrimped in situ at the time of use, by expansion of the stent itself. Other optional techniques for applying the tissue cover include wrapping the tissue (e.g., when provided in the form of sheets or strips), and unrolling a tissue provided in a donut-like rolled configuration.

Covers of this invention can be adapted for use with any suitable stent, including presently available stents such as those available from AngioDynamics (“AngioStent), AVE (“Micro Stent”), Biotronik (“Biotronik Stent”), Boston Scientific/Medinol and Boston Scientific/SciMed (“NIR”), Cook (“GRI”), C. R. Bard (“Angiomed” and “X-Trode”), Global Therapeutics (“Freedom” and “Freedom Force”), Guidant/ACS (“Multi-Link”), Johnson & Johnson/Cordis (“Palmaz-Schatz”, “Crown”, “Crossflex”), Medtronic (“Witkor” and “BeStent”), Medtronic/Instent (“CardioCoil”), Pfizer/Schneider (“Wallstent”), and Progressive Angioplasty Systems/ACT (“AT-One”).

Such stents tend to be either self deployed (NIR, Angiomed, Cardiocol and WallStent brands) or deployed by balloon (as in the remaining designs specified above), and prepared of materials that include platinum/iridium (AngioStent), tantalum/silicon carbide (Biotronik), stainless steel (NIR/Medinol, X-Trode, Freedom and Freedom Force, Multi-Link, Palmaz Schatz, Crown, Crossflex, BeStent brands), nitinol (NR/SciMed, Angiomed, Cardiocoil, ACT-One brands), tantalum (Cordis and Wiktor brands, and multialloys (WallStent). The stents can be provided to have a variety of designs, including those selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations. See, for example “Market Pulse, Table 3 at www.med-device.com/tablethree.html, the disclosure of which is incorporated herein by reference.

In one preferred method, a tubular pericardium stent cover is provided by a method that involves both treating the tissue with an agent to reduct/eliminate BSE infectivity and at least one “dry peel” step, the overall method including the steps of:

1) obtaining pericardium from a suitable (e.g., USDA-approved) source,

2) cleaning the tissue and optionally, and preferably, treating it in order to reduce/eliminate potential BSE infectivity,

3) drying the tissue, e.g., by vacuum,

4) peeling off one or more layers of the tissue, in the dry state, to provide a tissue of desired thickness, and optionally air drying the peeled tissue in order to permit it to become further compact and thin,

5) optionally, forming the peeled tissue into tubular form (e.g., by adhering and/or suturing abutting or overlapping surfaces),

6) positioning the tissue on a mandrel or stent and moistening it in order to facilitate its crimping and forming,

7) optionally crosslinking the tissue in its crimped configuration,

8) optionally attaching the cover to the stent, e.g., by the use of one or more sutures, and

9) sterilizing and packaging the cover itself and/or the covered stent.

The invention will be further described by reference to the following non-limiting examples.

### EXAMPLES

### TEST PROCEDURES

Unless otherwise indicated, the various materials described herein were tested for physical integrity by standard tests using the general methods described below. These tests were performed on the ChemDyne MC1000 (Columbia Labs, Inc.) tensile testing system. Suture retention were tested according to American National Standard for Cardiovascular implants-Vascular prostheses from the Association for the Advancement of Medical Instrumentation (1994). Shrink temperature, circumferential tensile strength and suture retention were measured to test the physical integrity of the stent covers.

Moisture Content

Moisture content was analyzed on a Mettler Toledo HG53 Halogen Moisture Analyzer. A temperature setting of 200° C. was used. Results are recorded in a % moisture content. Moisture content is determined after vacuum drying and after air drying.

Suture Retention

The suture retention test determines the force necessary to pull a suture from the prosthesis. Suture retention was performed using 5-0 Prolene suture. The needle was placed into the tissue with a 2 mm bite below the edge of the tissue. The suture is pulled at a constant force moving up at 100 mm/min, sampling at 20 Hz. Suture retention was performed on the ChemDyne MC1000 (Columbia Labs, Inc.) tensile testing system. Suture retention was tested according to American National Standard for Cardiovascular implants-Vascular prostheses from the Association for the Advancement of Medical Instrumentation (1994).

Stress Strain

Stress-strain was performed on the ChemDyne MC1000 (Columbia Labs, Inc.) tensile testing system. The stress-strain test gives unidirectional tensile properties such as tensile strength, strain at break, and Young's Modulus.

Shrink Temperature

Shrink temperature is the temperature at which the collagen denatures. The shrink temperature was measured using a 30 gram preload in a bath of water at steadily increasing temperature. The shrink temperature was calculated by using the knee method. The shrink temperature is dependent upon the cross-linking agent used.

Circumferential Tensile Strength

Circumferential tensile strength was measured with the cover in its tubular form. One 3 mm segment was placed onto two rounded pins. It was then stretched at 50 mm/min until the break point was reached. Circumferential tensile strength was defined as the peak load divided by twice the lengthin N/mm, force/2*L. Measurements were also made in terms of displacement vs. force.

### EXAMPLE 1

### Human Umbilical Cord Artery (“HUCA”) Stent Cover

A cylinder of tissue is constructed from human umbilical cord artery which is crimped into an expandable stent cover.

Cleaned fresh human umbilical cord arteries (HUCA) were obtained from hospital wards, rinsed with water and stored frozen until use. The tissues were rinsed in ultra-filtered deionized (UFDI) water and placed into 2 L of UFDI water which was changed each day Monday through Friday for 2 weeks. Arteries were isolated from the cord by inserting a 2 mm diameter mandrel into the arteries and carefully stripping away the rest of the cord. Excess tissue was gently removed from the arteries. Arteries prepared in this manner were stored in 70% ethanol at 4 C. until further processing.

Once isolated from the surrounding tissue, the HUCA were placed onto 3 mm to 4 mm diameter polyflorotetraethylene (PTFE) mandrels. Once positioned on the mandrels, the HUCA stent covers were fixed by placing them into a solution containing 0.25% glutaraldehyde (Electron Microscopy Sciences,) 0.9% NaCl (Fisher,) and 0.02% sodium bicarbonate (Sigma) at pH 7.4-8.4 for 48 hours. The tissues were then removed from the PTFE mandrels and placed into UFDI water.

The fixed tissues were cut into 30 mm segments, and a 2 mm cross-section was taken from one end to be used for wall thickness measurement. The segments were then placed onto 1.5 mm diameter PTFE mandrels. The tissue was carefully dampened and crimped onto the smaller mandrels using a rolling motion on a low lint cloth. Each crimp was very small, almost undetectable, giving the appearance of a congruent surface on the 1.5 mm mandrels. Once the crimping was complete, the covers were air dried and sterilized by placing them into a solution of 70% EtOH and 1% propylene oxide for 2 weeks, after which they were placed into a 1% propylene oxide storage solution.

Results are provided in TABLE 1 below. These results are consistent with data on human umbilical cord vein cross-linked in 1% glutaraldehyde.

The present stent cover can be placed over a stent in the operating room or in conjunction with a stent prior to packaging. The stent cover is designed to be expandable over the stent, by crimping and drying the tissue. Once crimped, the tissue can be stretched in situ and will return close to the original uncrimped conformation. This is beneficial, giving a close fit onto the stent. This stent cover, made from human umbilical cord artery, is extremely thin, adding less than 200 μm of thickness to the stent diameter. Applicant's experience with arterial and vascular prostheses formed of glutaraldehyde cross-linked tissue would indicate that no tissue-blood surface related problems are expected. The water content of the dry tissue is preferably about 5% to about 10% by weight based on the weight of the tissue.

TABLE 1
Test Results of HUCA Stent Cover

| | | Shrink | Circumferential |
|---|---|---|---|
| | Suture Retention | Temperature | Tensile Strength |
| Test Parameters | (g) | (C) | (N/mm) |
| Mean | 41.9 | 81.8 | 0.403 |
| St. Dev. | 20.3 | 1.7 | 0.177 |
| No. of Samples | 5 | 6 | 5 |

### EXAMPLE 2a

### Thin Bovine Pericardium Stent Cover

A cylinder of tissue is constructed from thin bovine pericardium which is crimped into an expandable stent cover.

Bovine pericardium was obtained in the U.S. from USDA-inspected healthy cows, minimum age 12 months. Fresh pericardium was obtained from and sent through a series of three saline rinses, followed by a final ice cold water rinse. Excess liquid was squeezed out of the tissues, and they were stored at 0 C. to 4 C. overnight for processing the following day. Tissue was then used fresh or stored at −20 C. Pericardium was then stripped of extraneous tissue and cut into 10 cm×10 cm squares for further processing.

Sodium Hydroxide Treatment

The tissue was placed into 1 M NaOH for 1 hour, using 200 ml of 1 M NaOH per pericardial sac. After 1 hour the tissue was rinsed in UFDI water and placed into a 50 mM citrate buffer, 200 ml buffer per sac. Buffer was changed every hour for 3 hours. Once the tissue was at a pH of 6.5-7.5 it was placed in a water bath over night.

Vacuum Drying/Peeling

The tissue was then placed in wire mesh racks and vacuum dried until the tissue had a water content of less than 15% moisture. Tissue was dried in a Virtis Genesis vacuum dryer at 115 mTorr. The tissue was trimmed on each side and the shiny side was peeled down to a thickness of less than 250 μm.

Air Drying

The tissue was wetted with UFDI (ultrafiltered deionized) water and placed shiny side down onto a clean transparency film or other very smooth surface. When allowed to air dry the tissue had a moisture content of less than 18% water and had become compacted, in the course of drying, to a final thickness of less than 100 μm.

Testing

The various tests described above were performed, the results of which are provided in TABLES 2a and 2b below, and were consistent with other glutaraldehyde crosslinked BioVascular, Inc. pericardium products. The exemplified method includes peeling the pericardium tissue while it is dry and then re-hydrating it and air-drying it. These steps allow for the tissue to be much thinner than if it were not peeled. In addition to the thinness of the tissue, the tissue also retains the strong physical characteristics of bovine pericardium.

Stent formation

Pericardium tissue prepared as described above was cut 30 mm long and 16 mm wide, to make a 4 mm diameter tube (30 mm long) with a 2 to 3 mm overlap for adhering and of varying diameters with 2 mm overlap for adhering. A gelatin suspension was made using porcine gelatin granules (Sigma) and 0.1% acetic acid solution (Aldrich.) The tissue was positioned shiny side up against a PTFE mandrel and each edge was coated with the gelatin suspension. Tissue edges were adhered together by gently rolling the mandrel and the covers were set to dry.

Once the tubes were dry, they were removed from the PTFE mandrels and placed onto a 1.5 mm diameter PTFE mandrels. The tissue was carefully dampened and crimped onto the smaller mandrels using a rolling motion on a low lint cloth. Each crimp was very small, almost undetectable, giving the appearance of a congruent surface on the 1.5 mm mandrels. Once the crimping was complete, the covers were air dried.

Dried crimped stent covers were placed into a 0.25% glutaraldehyde (Electron Microscopy Sciences,) 0.9% NaCl (Fisher,) 0.02% sodium bicarbonate (Sigma) at pH 7.4-8.4 for 48 hours. After fixation, the covers were rinsed with deionized water and placed into 70% EtOH and 1% proplyene oxide solution for 2 weeks and then into a 1% propylene oxide storage solution.

TABLE 2a
Test Results of BP Stent Cover

| | | Shrink | Circumferential |
|---|---|---|---|
| | Suture Retention | Temperature | Tensile Strength |
| Test Parameters | (g) | (C) | (N/mm) |
| Mean | 64.7 | 83.8 | 0.33 |
| St. Dev. | 29.7 | 1.3 | 0.23 |
| No. of Samples | 6 | 6 | 5 |

TABLE 2b
Stress-Strain Test of BP Sheet Form Used For Stent Cover

| | | Tensile | Strain | |
|---|---|---|---|---|
| | Thickness | Strength | at Break | Young’s Modulus |
| Test Parameters | (mm) | (MPa) | (%) | (MPa) |
| Mean | 0.088 | 9.6 | 16.7 | 43.8 |
| St. Dev. | 0.030 | 2.9 | 4.1 | 17.1 |
| No. of Samples | 6 | 6 | 6 | 6 |

A thin bovine pericardium stent cover, prepared in this manner, can be placed over a stent in the operating room. The present stent cover is designed to be expandable over the stent, e.g., by crimping the tissue prior to glutaraldehyde fixation. Once crosslinked, the tissue can be stretched and will return to the original crimped conformation. This is beneficial, giving a close fit onto the stent. This stent cover, made of peeled bovine pericardium, is extremely thin, adding less than 200 μm of thickness to the stent diameter.

### COMPARATIVE EXAMPLE 2b

### Preparation of Wet Peeled Bovine Pericardium

Bovine pericardium was peeled in its hydrated (“wet”) state in order to test its physical properties and compare them to those described for the dry-peeled pericardium described in Example 2a.

Hydrated pericardium was prepared in the following manner:

1) Fresh bovine pericardium was obtained in the same manner as described above,

2) The pericardium was peeled by grasping one edge of one corner of a rectangular piece of tissue (approximately 10 cm by 6 cm) with a forceps and grasping the other edge of the same corner with another forceps, and gently pulling the tissue apart.

3) The peeled tissue was placed into a solution of 0.25% glutaraldehyde, 0.9% NaCl and 0.02% sodium bicarbonate, in order to crosslink the tissue for 42 hours at room temperature. Tissue was approximately 100 to 400 microns thick after crosslinking.

4) Physical parameters were determined and are summarized in TABLE 2c below.

TABLE 2c
Stress-Strain Test Results of Wet Peeled Bovine Pericardium

| | | Tensile | Strain | |
|---|---|---|---|---|
| | Thickness | Strength | at Break | Young’s Modulus |
| Test Parameters | (mm) | (MPa) | (%) | (MPa) |
| Mean | 0.23 | 6.1 | 34.5 | 9.2 |
| St. Dev. | 0.09 | 3.7 | 24.9 | 7.5 |
| No. of Samples | 6 | 6 | 6 | 6 |

It can be seen that the physical characteristics of the dry-peeled pericardium are significantly better, on balance, than those of the wet-peeled tissue, particularly in terms of thickness and strength (e.g., Young's Modulus).

### EXAMPLE 3

### Porcine Peritoneum Stent Cover

A cylinder of tissue was constructed from porcine peritoneum and crimped into the form of an expandable stent cover.

Cleaned fresh porcine peritoneum was obtained from 2 week old pigs. The tissue was taken from the inferior area of the peritoneal cavity, rinsed in deionized water and stored in 50% ethanol for shipment.

The tissue was rinsed in ultra-filtered deionized (UFDI) water and cleaned of extraneous fatty tissue and edges trimmed. The tissue was then placed into a 2:1 solution of ethyl acetate and methanol (Fisher) for 48 hours. Tissue was then rinsed in UFDI water for at least 24 hours. Shiny sides were placed into UFDI water and then placed shiny side down on a transparency film to air dry in a class 10,000 clean room environment. The tissue was cut 2.5 cm long and 16 mm wide to make 4 mm diameter tubes. A gelatin suspension was made using gelatin granules (Sigma) and 0.1% acetic acid solution (Aldrich.) The tissue was positioned shiny side up against a PTFE mandrel and each edge was coated with gelatin suspension. Tissue edges were adhered together by gently rolling the mandrel. The covers were set to dry.

Once the 4 mm tubes were dry, they were removed from the PTFE mandrels and placed onto a 1.5 mm diameter PTFE mandrels. The tissue was carefully dampened and crimped onto the smaller mandrels using a rolling motion on a low lint cloth. Each crimp was very small, almost undetectable, giving the appearance of a congruent surface on the 1.5 mm mandrels. Once the crimping was complete, the covers were air dried. Dried crimped stent covers were placed into a 0.25% glutaraldehyde (Electron Microscopy Sciences,) 0.9% NaCl (Fisher,) 0.02% sodium bicarbonate (Sigma) at pH 7.4-8.4 for 48-72 hours. After fixation, the covers were rinsed with saline and placed into 70% EtOH and 1% propylene oxide solution for 2 weeks and then into a 1% propylene oxide storage solution.

Results of these tests are provided in TABLE 3 below, and were consistent with other glutaraldehyde cross-linked BioVascular, Inc. products.

TABLE 3
Test Results of PP Stent Cover

| | | Shrink | Circumferential |
|---|---|---|---|
| | Suture Retention | Temperature | Tensile Strength |
| Test Parameters | (g) | (C) | (N/mm) |
| Mean | 126.3 | 88.9 | 0.362 |
| St. Dev. | 83.4 | 0.8 | 0.172 |
| No. of Samples | 10 | 4 | 5 |

A stent cover has been developed which can be placed over a stent in the operating room. The stent cover has been designed to be expandable over the stent. The tissue is crimped prior to going into glutaraldehyde crosslinking solution. Once crosslinked, the tissue can be stretched and will return to the original crimped conformation. This is beneficial, giving a close fit onto the stent. This stent cover, made porcine peritoneum, is extremely thin, adding less than 200 μm of thickness to the stent diameter.

What is claimed is:

1. A stent cover comprising a natural tissue dimensioned and adapted to be placed over an expandable stent and to be expanded in situ, upon expansion of the stent, in a manner that permits the cover to maintain its integrity and decrease the incidence and/or extent of intimal hyperplasia, as compared to a stent lacking the cover.

2. A stent cover according to claim 1 wherein the natural tissue is a strong tissue of mammalian origin and is sufficiently thin to permit the collapsed stent to be positioned in situ, and sufficiently biocompatible for prolonged use in vivo.

3. A stent cover according to claim 2 wherein the stent cover provides a thickness of less than about 250 microns.

4. A stent cover according to claim 3 wherein the stent cover provides a thickness of less than about 100 microns.

5. A stent cover according to claim 2 wherein the natural tissue is selected from the group consisting of mammalian arteries, fibro-serous membranes and serous membranes.

6. A stent cover according to claim 5 wherein the mammalian arteries comprise umbilical arteries, the fibro-serous membranes comprise pericardium, and the serous membranes comprise peritoneum.

7. A stent cover according to claim 1 wherein the stent cover is provided in a permanent form by a method that comprises chemically crosslinking the tissue.

8. A stent cover according to claim 1 wherein the cover is provided in the form of flat or textured sheets, strips or in tubular form.

9. A stent cover according to claim 8 wherein the cover is provided in tubular form.

10. A stent cover according to claim 9 wherein the cover is provided in a seamless tubular configuration.

11. A combination comprising a stent covered with a stent cover according to claim 1.

12. A combination according to claim 11 wherein the stent is selected from the group consisting of self deployed stents and balloon deployed stents.

13. A combination according to claim 12 wherein the stent is prepared from a material selected from the group consisting of platinum/iridium, tantalum/silicon carbide, stainless steel, nitinol, tantalum, and multialloys.

14. A combination according to claim 13 wherein the stent is provided in a design selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations.

15. A combination according to claim 11 wherein the stent cover provides a thickness of less than about 250 microns and the natural tissue is selected from the group consisting of mammalian arteries, fibro-serous membranes and serous membranes.

16. A combination according to claim 15 wherein the stent cover provides a thickness of less than about 100 microns and the mammalian arteries comprise umbilical arteries, the fibro-serous membranes comprise pericardium, and the serous membranes comprise peritoneum.

17. A combination according to claim 16 wherein the stent cover is provided in a permanent form by a method that comprises chemically crosslinking the tissue.

18. A combination according to claim 11 wherein the cover is provided in the form of flat or textured sheets, strips or in tubular form.

19. A combination according to claim 11 wherein the stent is selected from the group consisting of self deployed stents and balloon deployed stents, the stent is prepared from a material selected from the group consisting of platinum/iridium, tantalum/silicon carbide, stainless steel, nitinol, tantalum, and multialloys, the stent is provided in a design selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations and the stent cover provides a thickness of less than about 250 microns and the natural tissue is selected from the group consisting of mammalian arteries, fibro-serous membranes and serous membranes.

20. A combination according to claim 19 wherein the stent cover provides a thickness of less than about 100 microns and the mammalian arteries comprise umbilical arteries, the fibro-serous membranes comprise pericardium, and the serous membranes comprise peritoneum.

21. A method of preparing a stent cover according to claim 1, wherein the cover is prepared from pericardium, the method comprising obtaining pericardium tissue from a suitable source, cleaning the tissue, initially drying the tissue, peeling off layers of the tissue one or more times, in the dry state, to provide a tissue of desired thickness, positioning and forming the tissue to the shape of a mandrel or stent to form a stent cover, and sterilizing and packaging the cover and/or the covered stent.

22. A method according to claim 21 wherein the cleaned tissue is treated to reduce or eliminate potential BSE infectivity, followed by an initial drying step performed by vacuum.

23. A method according to claim 21 wherein the peeled tissue is air dried in order to permit it to become further compact and thin.

24. A method according to claim 21 wherein the peeled tissue is formed into tubular form.

25. A method according to claim 24 wherein the tubular form is made by adhering and/or suturing abutting or overlapping surfaces of the peeled tissue.

26. A method according to claim 23 wherein the tissue is formed to the shape of the mandrel or stent by moistening and crimping the air dried, compacted tissue.

27. A method according to claim 21 wherein the cleaned tissue is treated to reduce or eliminate potential BSE infectivity, followed by an initial drying step performed by vacuum, and the peeled tissue is air dried in order to permit it to become further compact and formed into tubular form.

28. A method according to claim 27 wherein the tubular form is made by adhering and/or suturing abutting or overlapping surfaces of the peeled tissue and the tissue is formed to the shape of the mandrel or stent by moistening and crimping the air dried, compacted tissue.

29. A method according to claim 21 wherein the stent is selected from the group consisting of self deployed stents and balloon deployed stents, the stent is prepared from a material selected from the group consisting of platinum/iridium, tantalum/silicon carbide, stainless steel, nitinol, tantalum, and multialloys, the stent is provided in a desgin selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations and the stent cover provides a thickness of less than about 250 microns.

30. A method according to claim 29 wherein the resultant stent cover provides a thickness of less than about 100 microns.

31. A method of using a stent cover, the method comprising providing a stent and stent cover combination according to claim 11, implanting the stent in its collapsed form into a vessel and expanding the stent and cover in situ in order to provide permanent support for the vessel.

32. A method according to claim 31 wherein the stent is selected from the group consisting of self deployed stents and balloon deployed stents, the stent is prepared from a material selected from the group consisting of platinum/iridium, tantalum/silicon carbide, stainless steel, nitinol, tantalum, and multialloys, the stent is provided in a design selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations and the stent cover provides a thickness of less than about 250 microns and the natural tissue is selected from the group consisting of mammalian arteries, fibro-serous membranes and serous membranes.

33. A method according to claim 32 wherein the stent cover provides a thickness of less than about 100 microns. and the mammalian arteries comprise umbilical arteries, the fibro-serous membranes comprise pericardium, and the serous membranes comprise peritoneum.

34. A stent and stent cover combination according to claim 11, the combination being in expanded form with the cover positioned in apposition to the interior surface of a vessel.

35. An expanded combination according to claim 34 wherein the stent is selected from the group consisting of self deployed stents and balloon deployed stents, the stent is prepared from a material selected from the group consisting of platinum/iridium, tantalum/silicon carbide, stainless steel, nitinol, tantalum, and multialloys, the stent is provided in a design selected from the group consisting of single wire sinusoidals with longitudinal spines, geometric struts, slotted tubes with articulations, flexible coils, flexible coils with flat struts and an axial spine, coils, single wire fishbones, multiple links with articulations, slotted tubes with spiral articulations, sinusoidal helical coils, single wire sinusoidal helicies, sinusoidal helical coils, serpentine meshes, spiral coils, multiple wire brails, and slotted tubes with single articulations and the stent cover provides a thickness of less than about 250 microns and the natural tissue is selected from the group consisting of mammalian arteries, fibro-serous membranes and serous membranes.

36. An expanded combination according to claim 35 wherein the stent cover provides a thickness of less than about 100 microns. and the mammalian arteries comprise umbilical arteries, the fibro-serous membranes comprise pericardium, and the serous membranes comprise peritoneum.
